(19) United States
(12) Patent Application Publication
(10) Pub. No.: US 2009/0171148 A1
(43) Pub. Date: Jul. 2, 2009

(54) Capsule endoscope system having a sensing and data discriminating device and discrimination method thereof

(76) Inventors: Shih-Chieh Lu, Hsinchu City (TW); Mang Ou-Yang, Hsinchu City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/005,291

(22) Filed: Dec. 27, 2007

(51) Int. Cl. A61B 1/04

(52) U.S. Cl. 600/109; 600/118

(57) ABSTRACT

A capsule endoscope system having a sensing and data discriminating device and the discrimination method thereof are proposed. After a sensor captures data, a data processor performs the extraction of a characteristic value from the data and then comparison and discrimination. Data are then divided into two types: one to be transmitted and the other not to be transmitted. The redundancy of data transmission can be reduced, the amount of data transmission can be lowered, and the effect of power saving can also be accomplished.

### BACKGROUND OF THE INVENTION

1. Field of the Invention

The present invention relates to a capsule endoscope system and, more particularly, to a capsule endoscope system having a sensing and data discriminating device and the discrimination method thereof.

2. Description of Related Art

U.S. Pat. No. 5,604,531 disclosed a capsule endoscope. When examining the gastrointestinal tract of a patient, the patient has to wear a receiver first. After the patient swallows a capsule endoscope, light from an LED light source will illuminate the gastrointestinal tract and be reflected to a CMOS component in the capsule endoscope. An image transmission module then transmits the image of the gastrointestinal tract obtained by an image capturing module to the external receiver. After the examination of the gastrointestinal tract is finished, the capsule endoscope passes the gastrointestinal tract and is then expelled along with excrement.

In the prior art capsule endoscope, light from an LED light source illuminates an object and is then reflected to a CMOS component. An image transmission module then transmits the image of the gastrointestinal tract obtained by the image capturing module to an external receiver. Because this endoscope capture images continuously and each image frame is transmitted to the external receiver, the battery power is constantly consumed. It may occur that the battery power is used up before the examination is finished. In the taken image frames, there are usually many similar or repeated images, which will consume much battery power to cause a too early drainage of the battery power.

In addition to image data, there are also applications in pH meters and temperature sensors in the prior art. The present invention aims to propose a capsule endoscope system having a sensing and data discriminating device and the discrimination method thereof to solve the above problems in the prior art.

### SUMMARY OF THE INVENTION

An object of the present invention is to provide a capsule endoscope system having a sensing and data discriminating device and the discrimination method thereof, in which a data processor performs the extraction of a feature value of data and then comparison and screening so that the required data can be obtained while screening out redundant data.

Another object of the present invention is to provide a capsule endoscope system having a sensing and data discriminating device and the discrimination method thereof, in which image estimation is achieved by the extraction of a feature value and then comparison and screening to determine whether to transmit or not. If the consumed power of the added digital circuit is lower than the consumed power of redundant transmission, the proposed method has the advantage of power saving.

To achieve the above objects, the present invention provides a capsule endoscope system having a sensing and data discriminating device and the discrimination method thereof. The capsule endoscope system comprises a sensor, a data processor; and an RF transmitter. The sensor can be an image sensor, a temperature sensor, a pressure sensor, a position sensor or a pH meter. The present invention will be exemplified below with an image sensing and data discriminating device. The capsule endoscope system having a sensing and data discriminating device comprises an image sensing and data discriminating device, a data processor and an RF transmitter. The image sensing and data discriminating device captures a sequence of images. The data processor then processes the image data through the extraction of a feature value to decide image variation. After comparison and screening, the image data is divided into two types: one to be transmitted and the other not to be transmitted. If a data transmission signal is obtained, the image is outputted to an external receiver via the RF transmitter; if a no-data-transmission signal is obtained, the image won't be sent out via the RF transmitter.

### BRIEF DESCRIPTION OF THE DRAWINGS

The various objects and advantages of the present invention will be more readily understood from the following detailed description when read in conjunction with the appended drawing, in which:

FIG. 1 is a system block diagram of the present invention; and

FIG. 2 is a flowchart of the present invention.

### DETAILED DESCRIPTION OF THE PREFERRED EMBODIMENTS

Capsule endoscopes developed in recent years have replaced conventional tubular fiber endoscopes. This is a big advance in the medicine, and lessens patients' pains at the same time. After a patient swallows a capsule endoscope into his mouth, the capsule endoscope will go along the esophagus, the stomach to small intestines and then the large intestine, and is finally expelled along with excrements. Meanwhile, the capsule endoscope will constantly capture images and transmit the captured images to a receiver worn at his belly, thereby obtaining image data of the gastrointestinal tract.

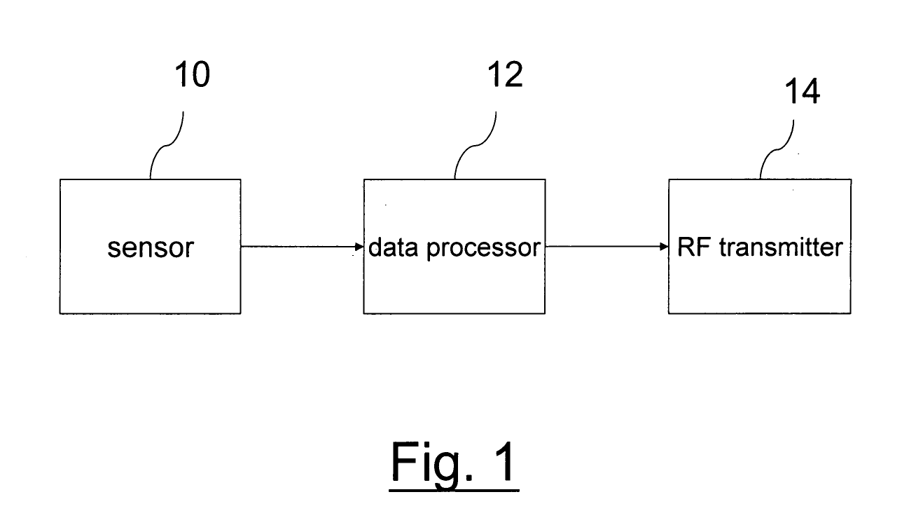

The present invention will be exemplified below with an image sensing data discriminating device. As shown in FIG. 1, a capsule endoscope system having a sensing and data discriminating device of the present invention comprises a sensor 10, a data processor 12, and an RF transmitter 14. The sensor 10 captures a sequence of images. The data processor 12 processes and discriminates image frames. In the data processor 12, each frame data can be raw data or compressed data. If the frame data is raw data, characteristic values of two consecutive frames can be based on for the determination of image similarity. This characteristic value can be an RGB or a CMY signal, or a brightness (Y) or color (C) signal, or another image character obtained from these signals. On the other hand, if the frame data is compressed data, compression technique like MPEG can be used (e.g., I, B and P frames have an entropy value for deciding image variation) as the basis for the determination of image similarity. No matter the frame is compressed image or raw image, the data processor 12 will discriminate the image to get a decision value. Through comparison of this decision value and a predetermined threshold, the data is determined to be transmitted or not to be transmitted. The threshold can be a single value or a range value. The RF transmitter 14 then transmits the data to be transmitted to an external receiver.

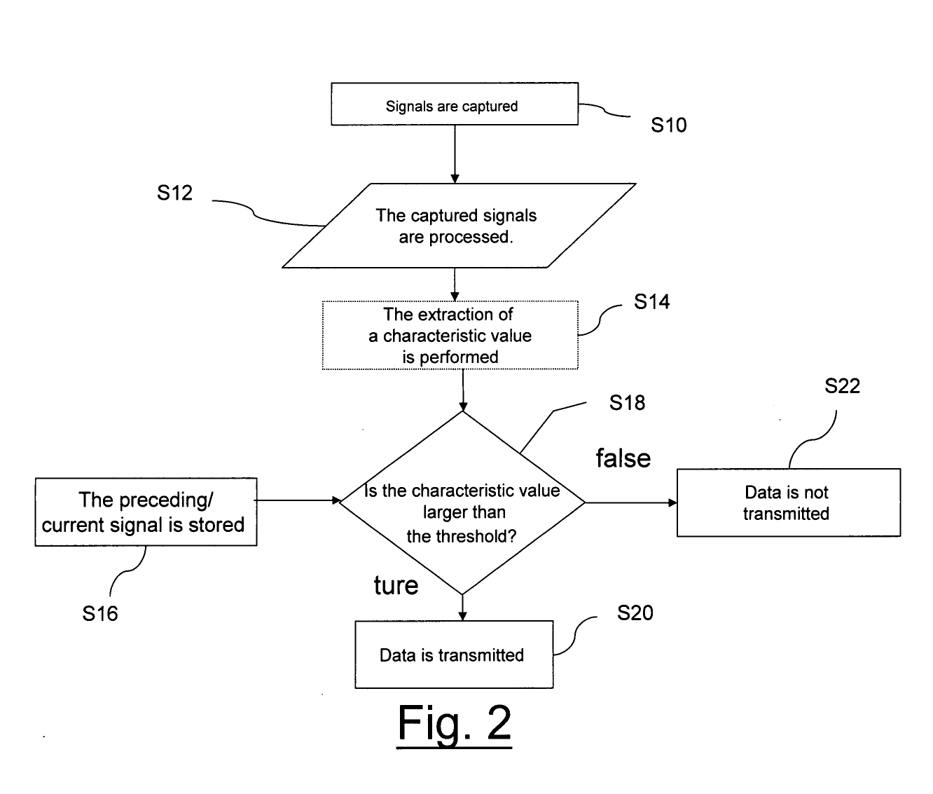

FIG. 2 is a flowchart of the present invention. As shown in FIG. 2, the sensor 10 captures a sequence of images (Step S10). The sensor 10 can be a CCD image sensor or a CMOS image sensor. The data processor 12 then processes the captured images (Step S12). The captured images can be raw data or compressed data. Next, if the frame data is raw data, characteristic values of two consecutive frames can be based on for the determination of image similarity. This feature value can be an RGB or a CMY signal, or a brightness (Y) or color (C) signal, or another image character obtained from these signals. On the other hand, if the frame data is compressed data, compression technique like MPEG can be used to get an entropy value of I, B or P frame for deciding image variation, and a threshold is compared with the entropy value to determine image similarity or difference (Step S14). A preceding image is temporarily stored in a buffer, and a current image data is constantly updated for comparison with the next image data; or a new image data will replace the old one to be stored in the buffer when this new image data has a different characteristic value. In this way, the image data can be constantly updated (Step S16). Subsequently, a preceding image and a current image are compared to decide image similarity or difference for determination of whether to transmit or not (Step S18). If the feature value of the image is larger than the threshold, the image is judged as a data to be transmitted, and the image data is transmitted to an external receiver via the RF transmitter 14 (Step S20). On the other hand, if the characteristic value of the image is smaller than the threshold, the image is judged as a data not to be transmitted, and won't be transmitted via the RF transmitter 14 (Step S22). The determination method can be any operation such as addition, subtraction, multiplication, division, and so on, and the judgment result can differ according to different logic conditions. For instance, the image is can be judged as a data to be transmitted if its feature value is smaller than the threshold.

The threshold can be a single value or a range value, and is used as the standard value for determination. The range of the threshold can be set according to demand. In the present invention, an image is first processed, and a decision of whether to transmit the image or not is then made. If the consumed power of the added digital circuit is lower than the consumed power of redundant transmission, the proposed method has the advantage of power saving. Moreover, in addition to an image sensor, the sensor of the present invention can also be a temperature sensor, a pressure sensor, a position sensor, or a pH meter.

Although the present invention has been described with reference to the preferred embodiment thereof, it will be understood that the invention is not limited to the details thereof. Various substitutions and modifications have been suggested in the foregoing description, and others will occur to those of ordinary skill in the art. Therefore, all such substitutions and modifications are intended to be embraced within the scope of the invention as defined in the appended claims.

I claim:

1. A capsule endoscope system having a sensing and data discriminating device capable of being placed inside human body to take signals, said capsule endoscope system comprising:
a sensor capable of capturing a plurality of signals;
a data processor capable of screening said plurality of signals to get at least one screened signal for processing; and
an RF transmitter for transmitting said screened signal to an external receiver.

2. The capsule endoscope system as claimed in claim 1, wherein said sensor is capture a sequence of signals.

3. The capsule endoscope system as claimed in claim 1, wherein said sensor is an image sensor, a temperature sensor, a pressure sensor, a position sensor, or a pH meter.

4. The capsule endoscope system as claimed in claim 3, wherein said image sensor is a CCD image sensor or a CMOS image sensor.

5. The capsule endoscope system as claimed in claim 1, wherein said signals are raw data or compressed data.

6. The capsule endoscope system as claimed in claim 5, wherein if said signal is raw data, feature values of two consecutive frames are based on for the determination of image similarity, and the feature value is an RGB or CMY signal, a brightness (Y) or color (C) signal, or an edge feature signal.

7. The capsule endoscope system as claimed in claim 5, wherein if said signal is compressed data, compression technique like MPEG is used to get an entropy value of I, B or P frame for deciding signal variation, and said entropy value is then used as the basis for determination of signal similarity.

8. The capsule endoscope system as claimed in claim 5, wherein signals are feasible to take only 7 to 1 most significant bits (MSB) of original 8 bits of said signal for processing to save gate-count or memory capacity.

9. The capsule endoscope system as claimed in claim 1, wherein a threshold for determination of said data processor is a single value or a range value.

10. The capsule endoscope system as claimed in claim 9, wherein said threshold is used as the standard value for comparing said data file to determine whether to transmit said data file or not.

11. A discrimination method of a capsule endoscope system comprising the steps of:
capturing a plurality of signals;
performing the extraction of a feature value of said plurality of signals and screening said plurality of signals by comparing a preceding signal and a current signal to determine whether there is difference, transmitting an output signal if the result is true or transmitting no output signal if the result is false; and
transmitting said screened signal as an output signal to an external receiver.

12. The discrimination method as claimed in claim 11, wherein said signals are raw data or compressed data.

13. The discrimination method as claimed in claim 12, wherein if said signal is raw data, feature values of two consecutive frames are based on for the determination of image similarity, and the characteristic value is an RGB or CMY signal, a brightness (Y) or color (C) signal, or the image characters e.g. edge, smooth etc.

14. The discrimination method as claimed in claim 12, wherein if said signal is compressed data, compression technique like MPEG is used to get an entropy value of I, B or P frame for deciding signal variation, and said entropy value is then used as the basis for determination of signal similarity.

15. The discrimination method as claimed in claim 11, wherein the extracted feature value is used as a threshold, and said threshold is a single value or a range value.

16. The discrimination method as claimed in claim 11, wherein said threshold is used as standard value for comparing said plurality of signal to determine whether to transmit or not.

17. The discrimination method as claimed in claim 11, wherein said current signal is stored in a buffer until a signal different from said current signal, and said new signal will then replace said current signal to be stored in said buffer, waiting for the incoming of next signal.

18. The discrimination method as claimed in claim 11, wherein signals are feasible to take only 7 to 1 most significant bits (MSB) of original 8 bits of said signal for processing to save gate-count or memory capacity.
